(19) United States
(12) Patent Application Publication
(10) Pub. No.: US 2007/0168229 A1
(43) Pub. Date: Jul. 19, 2007

(54) Health care network system using smart communicator and method thereof

(75) Inventor: Jong Hee Kim, Seongnam-si (KR)

Correspondence Address: CHA & REITER, LLC, 210 ROUTE 4 EAST STE 103, PARAMUS, NJ, 07652, US

(73) Assignee: Samsung Electronics Co.;LTD

(21) Appl. No.: 11/637,980

(22) Filed: Dec. 13, 2006

(30) Foreign Application Priority Data
Jan. 16, 2006 (KR) 2006-4275

(51) Int. Cl. G06Q 10/00; A61B 5/00

(52) U.S. Cl. 705/2; 600/300

(57) ABSTRACT

The present invention relates to a health care network system and a method thereof utilizing a smart communicator. The health care network system of the present invention includes: a hospital server for providing a prescription based on information of a user's health state and abnormality corresponding to the user's prescription request through the Internet; at least one biological state measuring sensor installed in at least one home instrument to detect the user's various biological states; a home server analyzing the user's health state based on the measured biological states, identifying and transmitting the health abnormality, and receiving and transmitting the prescription by connecting to the hospital server when the prescription is requested; and a smart communicator receiving the user's health state information and health abnormality information transmitted by the home server, transmitting the user's prescription request information to the home server when the prescription request is made, and receiving prescription information transmitted by the home server.

### CLAIM OF PRIORITY

This application claims priority to an application entitled “HEALTH CARE NETWORK SYSTEM USING SMART COMMUNICATOR AND METHOD THEREOF,” filed in the Korean Intellectual Property Office on Jan. 16, 2006 and assigned Serial No. 2006-0004275, the contents of which are incorporated herein by reference.

### BACKGROUND OF THE INVENTION

1. Field of the Invention

The present invention relates to a health care network system, and more particularly, to a health care network system and a method thereof for monitoring a user's biological states at home or at work place.

2. Description of the Prior Art

Presently, many people endeavor to care, conserve, and improve their health by utilizing non-medical facilities such as sports clubs in addition to diagnoses and medical facilities for the purpose of early detection or prevention of diseases. Additionally, various instruments supporting home health care are being developed for treatment of diseases at home.

It is well known that, in professional medical facilities such as hospitals and clinics, various instruments of detecting biological symptoms are available. Biological states measured by these instruments are transmitted to a host computer (or a workstation of a medical information management room) through a local area network (LAN) or telemeter, so that medical data can be centrally controlled.

Unless admitted into a professional medical care, patients must frequently visit the medical facilities for check ups. However, this may be a greater burden to certain people and considerable physical and mental fatigue may be induced.

In a conventional system of collecting biological states and receiving corresponding prescriptions from a professional medical institution, the biological states are measured by specific instruments, which may cause a psychological stress to patients, thus degrading the reliability of measured data. For example, in an electrocardiogram record or blood pressure measuring instruments, the measured data frequently show abnormal values as the object of the measurement is stressed or excited. As a result, the measurement must be repeated often.

Recently, a method has been proposed to install devices detecting biological symptoms at a patient's home, transmit obtained information on biological states to a host computer of a professional medical facility through a public communication line, and receive corresponding prescriptions. For example, there is a method of receiving prescription by installing a urinalysis instrument in a toilet bowl at home and transmitting the obtained urinalysis data to a remote computer. There is also a health care service method in which a terminal capable of measuring biological states such as blood pressure is disposed at a patient's home. The patient's blood pressure is measured at home according to an instruction from a central station. The measured data is transmitted to the central station, and the central station decides pharmaceutical dosing according to the transmitted data and then instructs the patient.

However, such a conventional health care service method performs a series of measuring programs that require a user's manipulation of a measuring instrument or a central station. Accordingly, there is a problem in that a user must be engaged in the operation of complicated measurement devices.

### SUMMARY OF THE INVENTION

The present invention provides a health care network system and a method thereof for collecting a user's biological state information and automatically providing health check information and prescription to the user.

Another aspect of the present invention is to provide such information in real time through an online system, thus eliminating the need to attend medical facilities.

Another aspect of the present invention is to provide a health care network system and a method thereof for collecting information on a plurality of biological states of a user daily and giving information on health solutions, such as health care, health conservation, and health improvement, to the user based on the collected biological states in the user's daily life.

In one embodiment, a health care network system according to an embodiment of the present invention includes: a hospital server for providing prescriptions based on a user's health state information and other health abnormality information corresponding to the user's request through the Internet; at least one biological state measuring sensor installed in at least one home instrument to detect the user's various biological states; a home server analyzing the user's health state based on the measured biological states, identifying and transmitting the health abnormality, and receiving and transmitting the prescription by connecting to the hospital server when the prescription is requested; and a smart communicator receiving the user's health state information and health abnormality information transmitted by the home server, transmitting the user's prescription request information to the home server when the prescription request is input and receiving prescription information transmitted by the home server.

The home server includes: a display unit for displaying information generated according to the operation of the home server; a communication unit for performing a mutual communication with at least one biological state measuring sensor, the smart communicator, and the hospital server; a health state analyzing unit for analyzing the user's health state based on the biological states; a control unit for identifying the user's health abnormality according to the result of the health state analysis, transmitting the user's health state information and health abnormality information to the smart communicator through the communication unit, and receiving the prescription by connecting to the hospital server through the communication unit and then transmitting the prescription to the smart communicator according to the prescription request from the smart communicator; and a storage unit for storing information on the user's biological states, the user's health state and abnormality, and the prescription.

The storage unit further stores information corresponding to the health abnormality and information on a basic health care guide as a basic guide for the user's health care and conservation.

The home server further includes a health care guide unit for comparing the result of the health state analyzed by the health state analyzing unit with information on basic health care stored in the storage unit, and generating information on a health care guide for the user's health care according to the information on a basic health care. The control unit transmits the health care guide information generated by the health care guide unit to the smart communicator.

The smart communicator includes: a communication unit for performing a mutual communication with the home server in a local area and/or in a wide area; a display unit for displaying information transmitted by the home server and information generated by the operation of the smart communicator; a storage unit for storing the health state information and prescription information transmitted by the home server; a key-input unit for command input of the prescription request; and a control unit for displaying and storing, on the display unit and in the storage unit respectively, the health state information and prescription information received through the communication unit, and transmitting information on a prescription request input by the key-input unit to the home server through the communication unit.

The communication unit includes: an IR (infrared) communication unit for communicating at home with the home server; a Wibro communication unit for providing a bi-directional wireless communication outside the home with the home server through a Wibro communication network; a Zigbee module for providing a function of local area wireless communication for home automation and data network of the home server; and a GPS (Global Positioning System) module for receiving information on longitude and latitude from satellites and identifying the user's current location.

The biological state measuring sensor includes at least one from the group consisting of: a body weight measuring sensor, body moisture measuring sensor, body fat measuring sensor, electrocardiogram measuring sensor, blood pressure/pulse rate measuring sensor, breathing rate measuring sensor, body height measuring sensor, body temperature measuring sensor, and exercise rate measuring sensor.

A health care method used in a health care network system utilizing a smart communicator according to the embodiment of the present invention includes: measuring a user's various biological states by utilizing biological state measuring sensors installed at home; analyzing the user's health state based on the measured biological states and identifying health abnormality; transmitting the health state information and health abnormality information to the user's smart communicator; receiving a prescription based on the health state information and health abnormality information by connecting to a registered hospital server, when prescription request information is received from the user's smart communicator; and transmitting the received prescription information to the user's smart communicator.

Preferably, the health care method further includes a step of storing the health state information and health abnormality information according to the result of the analysis, and a step of storing the prescription information received from the hospital server.

The biological state measuring sensor includes at least one from the group consisting of: a body weight measuring sensor, body moisture measuring sensor, body fat measuring sensor, electrocardiogram measuring sensor, blood pressure/pulse rate measuring sensor, breathing rate measuring sensor, body height measuring sensor, body temperature measuring sensor, and exercise rate measuring sensor.

According to the present invention, a home server collects information on biological states measured by sensors having individual measuring functions of the biological states and installed in home instruments, identifies the user's health state and abnormality, and transmits them to the user's smart communicator. If a prescription request is made from the smart communicator in the case of health abnormality, the home server receives a prescription based on the user's health state information and health abnormality information from a registered hospital server, and transmits the prescription to the smart communicator. Accordingly, information on the user's biological states may be collected more precisely in the user's daily life, from which speedy health checking and early detection of health abnormality are possible, and pharmaceutical prescription may more conveniently be provided according to the occurrence of health abnormality.

### BRIEF DESCRIPTION OF THE DRAWINGS

FIG. 1 is a block diagram showing a health care network system according to an embodiment of the present invention.

FIG. 2 is a view showing examples of home instruments installed with sensors having individual functions and enabling easier measurement of biological states in the health care network system of FIG. 1.

FIG. 3 is a detailed block diagram of the home server of FIG. 1.

FIG. 4 is a view showing various kinds of information stored in the storage unit of FIG. 3.

FIG. 5 is a detailed block diagram of the smart communicator of FIG. 1.

FIG. 6 is a flow chart showing a health care method utilizing the health care network system according to another embodiment of the present invention.

### DETAILED DESCRIPTION OF THE INVENTION

Hereinafter, embodiments of the present invention will be described in detail with reference to the accompanying drawings. The same reference number will be used for the same or like components in the accompanying drawings. For the purposes of clarity and simplicity, detailed explanations for well-known functions and compositions will be omitted for conciseness.

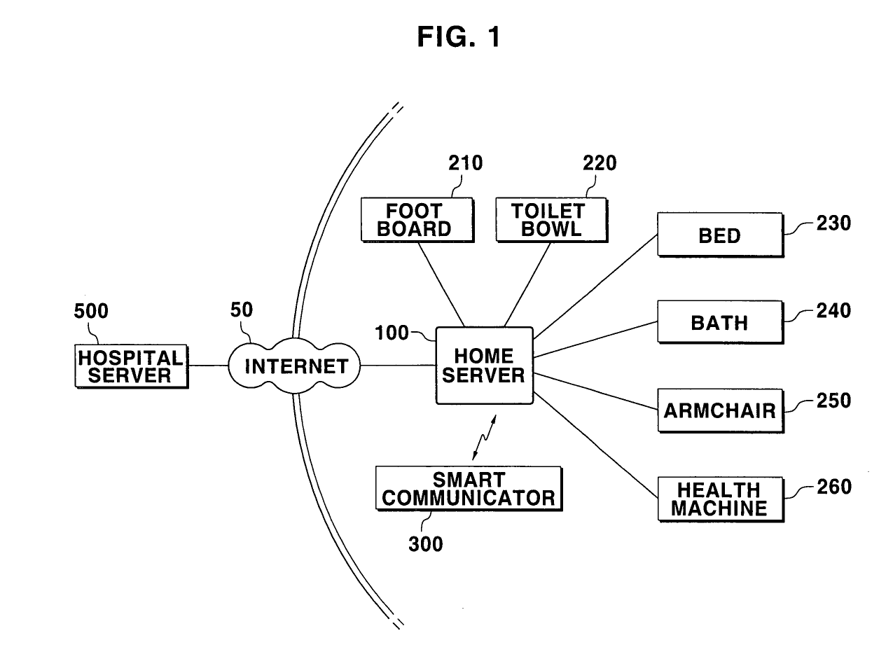

FIG. 1 is a block diagram showing a health care network system according to an embodiment of the present invention.

As shown in FIG. 1, the health care network system includes a home server 100, home instruments 210, 220, 230, 240, 250, and 260 equipped with sensors for measuring and collecting biological states with corresponding functions, a smart communicator 300, and a hospital server 500. The home instruments 210, 220, 230, 240, 250, and 260, installed with sensors for measuring the corresponding biological states, are instruments that a user utilizes daily at home. A footboard 210, toilet bowl 220, bed 230, bath 240, armchair 250, and health machine 260 are shown as examples of the home instruments according to the embodiment. However, it should be noted that the present invention can support other detecting devices, thus the number of instruments shown in FIG. 1 should not impose limitations on the scope of the invention.

The home server 100 controls the overall operation of home network systems installed at home and also processes a control command transmitted from the user's smart communicator 300 located at home or outside the home. The home server 100 stores information on a registered user received from one or more home instruments 210, 220, 230, 240, 250, and 260 and analyzes/checks the health state of the user.

The home server 100 transmits the analyzed health state information, including information relating to any abnormality found, to the smart communicator 300.

If there is a prescription request made by the user in advance or if an option to make such a request is presented to the user and responded via the smart communicator 300, the home server 100 connects to the hospital server 500 in charge of medical examination of the corresponding symptoms or diseases through the Internet 50, and then the home server 100 transmits the user's health state information and the found health abnormality information in order to receive an appropriate prescription from the hospital server 500.

Thereafter, the home server 100 stores the prescription information received from the hospital server 500 and transmits it to the smart communicator 300.

The smart communicator 300 has a function of controlling the operation of the home server 100 with a known communication method known in the art at home and/or outside the home, and utilizes a bi-directional communication with the home server 100 during operation. When the smart communicator 300 is located at home, it transmits/receives data to/from the home server 100 by performing a local area wireless communication. When the smart communicator 300 is located outside the home, it performs a mutual communication with the home server 100 through a wireless data communication network based on data communication, namely a Wibro communication network (not shown). Accordingly, the smart communicator 300 transmits control commands to home network instruments and the home server 100 as well as voice and image data through the wireless data communication network.

The smart communicator 300 is an information terminal for controlling/monitoring the home network instruments remotely and in real time through the home server 100 according to the user's control. The smart communicator 30 further supports wireless voice/image communication and mobile Internet. For this purpose, the smart communicator 300 may be equipped with a platform for driving Wibro, Zigbee, RFID (Radio Frequency Identification), IrDA (Infrared Data Association), and GPS (Global Positioning System) modules, and a user interface to control individual home network instruments.

The smart communicator 300 receives the health state information including the user's measured biological states from the home server 100, then displays and stores the same information. When any health abnormality information is received from the home server 100, the smart communicator 300 displays and stores the information, and identifies if the user has made or makes a prescription request. If there is a prescription request made from the user, the smart communicator 300 transmits the prescription request information to the home server 100.

The home server 100 transmits the health abnormality information and prescription request information to the hospital server 500 according to the request from the smart communicator 300. The hospital server 500 manages medical examination of diseases and issues prescriptions according to the user's health state, thus authorized to provide a prescription corresponding to the prescription request received through the Internet 50. The hospital server 500 provides the prescription for the corresponding disease based on the user's health state information and health abnormality information in response to the prescription request from the home server 100 through the Internet 50.

Thereafter, if the corresponding prescription information is received from the hospital server 500, the home server 100 stores the received prescription information and transmits it to the smart communicator 300.

The smart communicator 300 then displays and stores the prescription information transmitted by the home server 100. The prescription information may include information on a guideline for recovering from the health abnormality as well as a pharmaceutical prescription for the user.

As a result, the user may be provided with a health care instruction for recovering from the health abnormality and obtain prescribed drugs from a drug store by utilizing the prescription information received by the smart communicator 300.

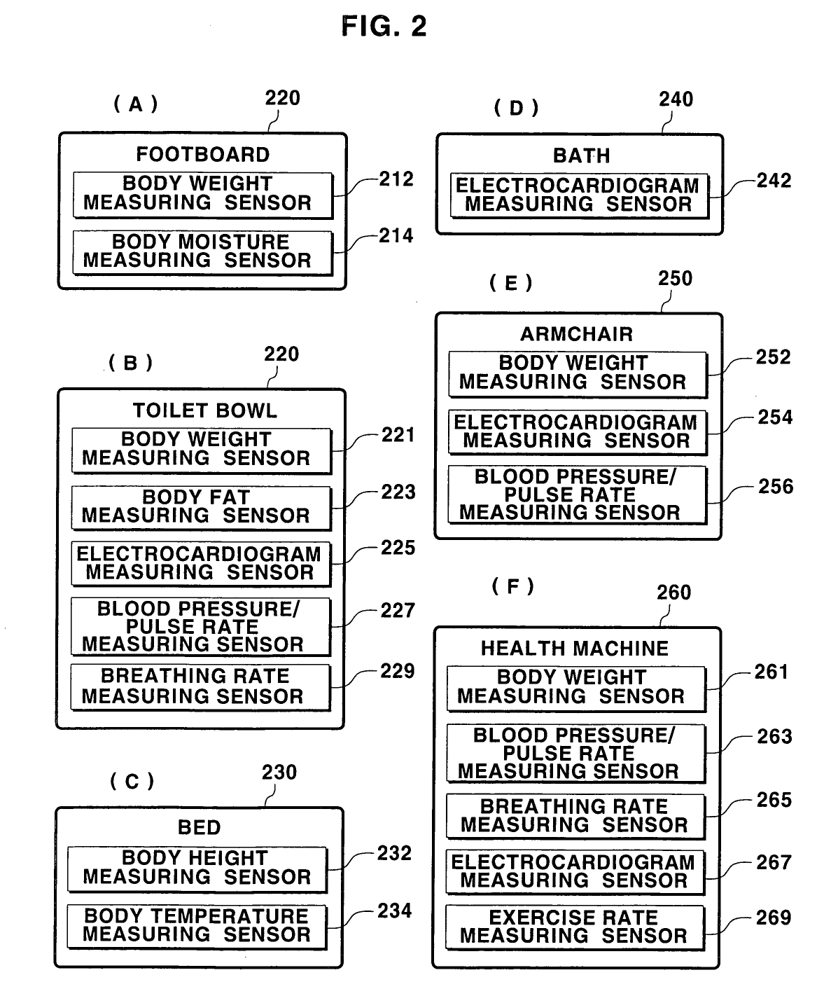

FIG. 2 shows examples of home instrument installed with different functions to enable an easier measurement of biological states in the health care network system of FIG. 1.

As shown in FIG. 2, (A) shows a footboard 210 including a body weight measuring sensor 212 and a body moisture measuring sensor 214.

(B) shows a toilet bowl 220 including a body weight measuring sensor 221, body fat measuring sensor 223, electrocardiogram measuring sensor 225, blood pressure/pulse rate measuring sensor 227, and breathing rate measuring sensor 229.

(C) shows a bed 230 including a body height measuring sensor 232 and a body temperature measuring sensor 234.

(D) shows a bath 240 including an electrocardiogram measuring sensor 242.

(E) shows an armchair 250 including a body weight measuring sensor 252, electrocardiogram measuring sensor 254, and blood pressure/pulse rate measuring sensor 256.

(F) shows a health machine 260 including a body fat measuring sensor 261, blood pressure/pulse rate measuring sensor 263, breathing rate measuring sensor 265, electrocardiogram measuring sensor 267, and exercise rate measuring sensor 269.

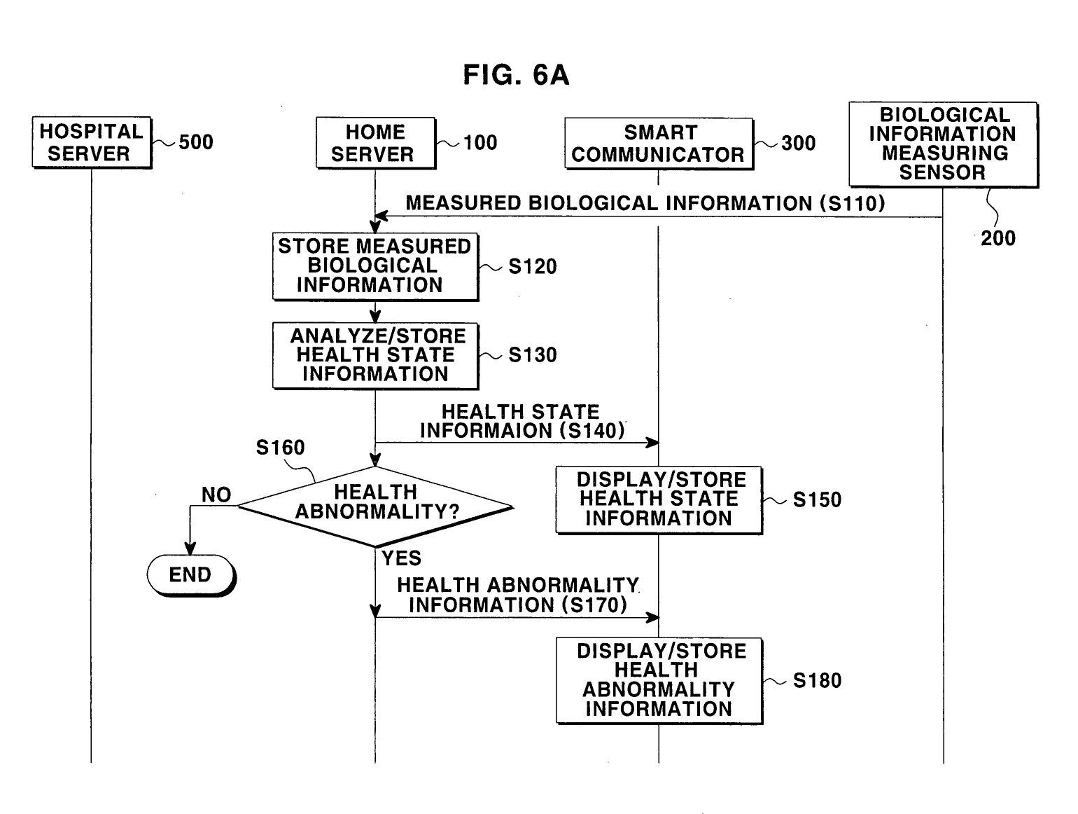

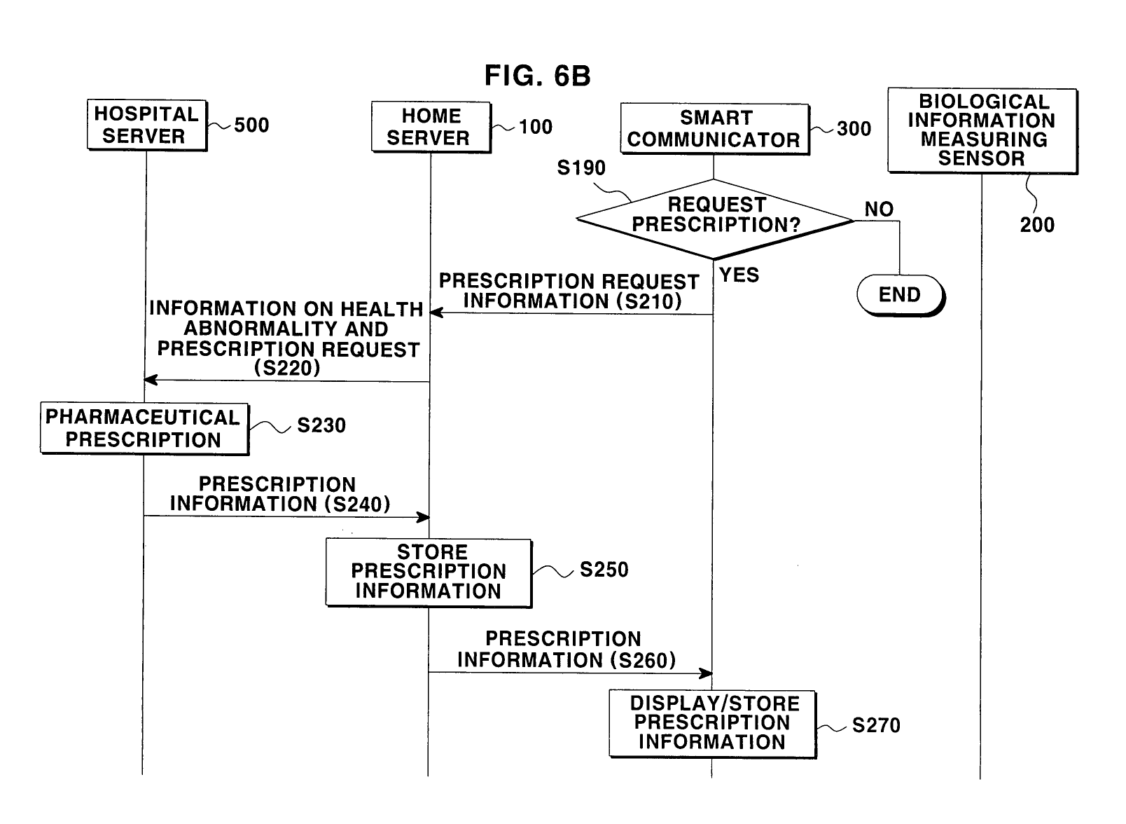

Biological information measuring sensors 200, which will be shown in FIG. 6A to 6B, include various sensors 212, 214, 221, 223, 225, 227, 229, 232, 234, 242, 252, 254, 256, 261, 263, 265, 267 and 269 as shown in FIG. 2.

The home instruments 210, 220, 230, 240, 250, and 260 individually transmit information on corresponding biological states of a user measured by the individual sensors to the home server 100.

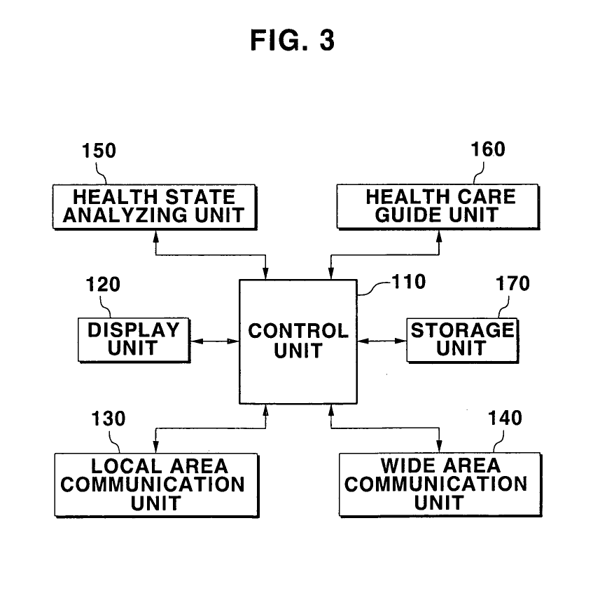

FIG. 3 is a detailed block diagram of the home server 100 of FIG. 1.

As shown in FIG. 3, the home server 100 includes a control unit 110, display unit 120, local area communication unit 130, wide area communication unit 140, health state analyzing unit 150, health care guide unit 160, and storage unit 170.

The control unit 110 controls the operation of the home server 100 and home instruments connected to the home server 100 by communication means such as a home network. The control unit 110 controls the operation of individual sensors installed in the home instruments 210, 220, 230, 240, 250, and 260, and collects biological state information measured by the corresponding sensors.

The display unit 120 displays status information according to the operation of the home server 100, and information transmitted from the instruments connected by communication means. Additionally, the display unit 120 displays the biological state information collected from the corresponding sensors.

The local area communication unit 130 provides a function of local area wired and/or wireless communication with the smart communicator 300 and home network instruments at home. The local area wireless communication unit 130 performs a local area wireless communication with the smart communicator 300 at home, and provides a function of performing a local area wired and/or wireless communication with the corresponding sensors. Accordingly, the local area communication unit 130 receives biological state information measured by the corresponding sensors.

The wide area communication unit 140 provides a function to communicate with a smart communicator located outside the home through a Wibro communication network.

The health state analyzing unit 150 analyzes/checks the user's health state based on the biological state information transmitted by the corresponding sensors. The control unit 110 transmits the checked user's health state information to the user's smart communicator 300. Additionally, if health abnormality is identified as a result of the analysis of the user's health state, the control unit 110 may include information on the health abnormality into the health state information and transmit it to the user's smart communicator 300.

The health care guide unit 160 compares the result of the health state analyzed by the health state analyzing unit 1 50 with predetermined basic health care information, and generates information to guide the user's health care according to the basic health care information. The control unit 160 transmits the generated health care guide information to the user's smart communicator 300.

The storage unit 170 stores programs required for a home networking operation of the home server 100 and stores information required for controlling the health care according to the embodiment of the present invention.

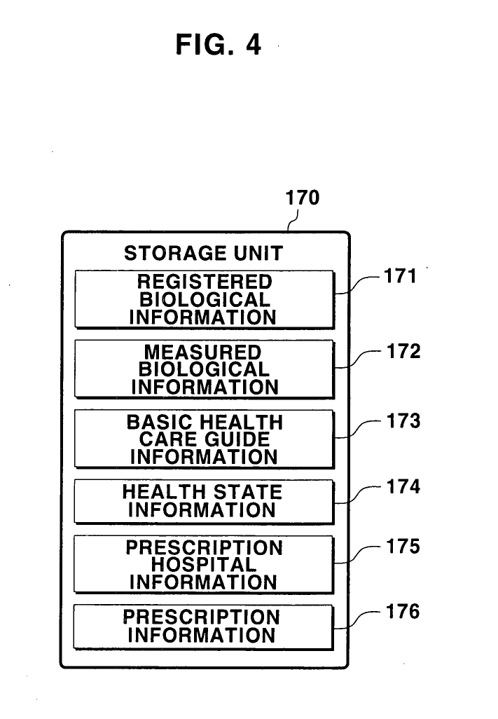

FIG. 4 shows various types of information stored in the storage unit 170 of FIG. 3.

As shown in FIG. 4, the storage unit 170 stores a user's pre-registered biological state information 171, biological state information 172 most recently measured by the corresponding sensors, pre-registered basic health care guide information 173, health state information 174 analyzed by the health state analyzing unit 150, prescription hospital information 175 having registered server information of the prescription hospital 500 corresponding to the types of health abnormality, and prescription information 176 received from the prescription hospital 500. The measured biological state information 172 is updated and stored with the registered biological state information 171 if a newly measured biological state information is received. It should be noted that other medical related information relating to a patient can be stored in the storage unit. Although a limited number of medical information is shown in FIG. 4 for illustrative purposes, it is to be understood that the storage unit 170 can support and store a much larger number of health related information. Thus, the number of data in the drawing should not impose limitations on the scope of the invention.

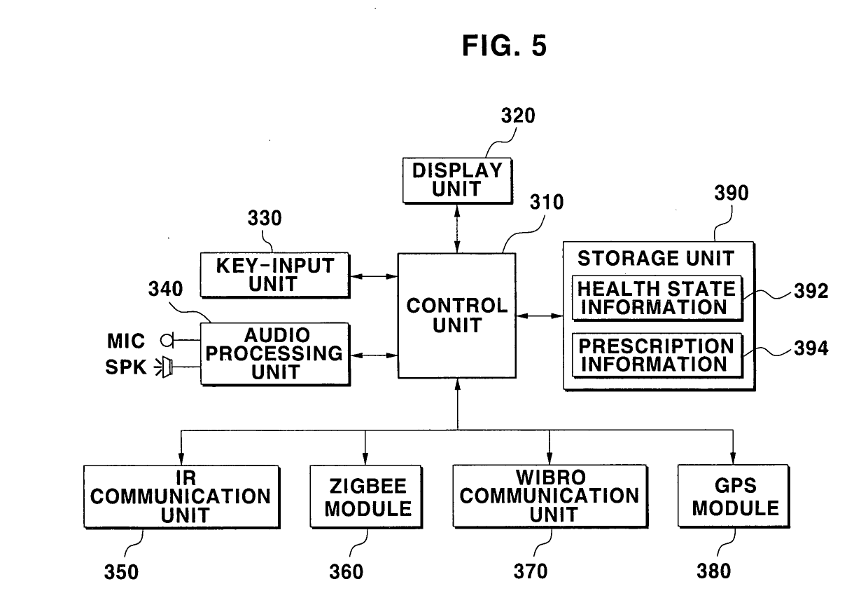

FIG. 5 is a detailed block diagram of the smart communicator 300 of FIG. 1.

As shown in FIG. 5, the smart communicator 300 includes a control unit 310, display unit 320, key-input unit 330, audio processing unit 340, IR communication unit 350, Zigbee module 360, Wibro communication unit 370, GPS module 380, and storage unit 390.

The control unit 310 controls the overall operation of the smart communicator 300, and remotely controls the home server 100 and home network instruments at home. The control unit 310 manages health state information including a user's biological state information transmitted by the home server 100, requests corresponding prescription to the home server 100 when health abnormality occurs or is detected, and manages prescription information provided corresponding to a prescription request. The control unit 310 may request the prescription to the home server 100 or directly to the hospital server 500 by connecting through the Wibro communication unit 370.

The display unit 320 displays information on operation status of the smart communicator 300, and displays the health state information and prescription information transmitted by the home server 100.

The key-input unit 330 inputs commands for the operation control of the smart communicator 300 through the control unit 310, and includes keys for controlling health care operation of the home server 100 by utilizing the corresponding sensors for measuring biological states, which are installed in the home instruments 210, 220, 230, 240, 250, and 260 according to the embodiment of the present invention. Additionally, the key-input unit 330 includes keys to manage the health state information analyzed from the user's biological states and received from the home server 100, prescription request, and prescription received from the hospital server 500.

The audio processing unit 340 outputs to the control unit 310 by converting analog audio signals input from a microphone to digital audio signals, and outputs through a speaker by converting digital audio signals input from the control unit 310 to analog audio signals under the control of the control unit 310.

The IR communication unit 350 controls the operation of the home server 100 in a local area IR wireless communication setting at home, and may receive/transmit data by utilizing mutual IR communication. The IR communication unit 350 is used to remote-control the operation of the home server 100 at home.

The Zigbee module 360 provides a function of local area wireless communication for home automation and data network.

The Wibro communication unit 370 provides a function of mobile wireless Internet communication and a function of bi-directional wireless communication with the home server 100 outside the home through the Wibro communication network. Therefore, the Wibro communication unit 370 can transmit/receive/identify short messages in communication with the home server 100 and control commands of the home server 100 and operation status information generated by the control of the home server 100. The Wibro communication unit 370 based on data communication may optionally be provided with a mobile communication protocol.

The GPS module 380 receives information on longitude and latitude from satellites, and is used to identify the user's current location.

The IR communication unit 350, Zigbee module 360, Wibro communication unit 370, and GPS module 380 may be selectively installed according to functional specification of the smart communicator 300.

The storage unit 390 stores programs required for the operation of the smart communicator 300 and operational configuration information of the home server 100. The storage unit 390 stores the programs required to control the health care through the home server 100 according to the embodiment of the present invention. Additionally, the storage unit 390 stores the user's health state information 392 and prescription information 394 provided by the home server 390. Such information may be displayed on the display unit 320 and transmitted to external instruments in a corresponding method according to the control of the control unit 310.

FIG. 6 is a flow chart showing a health care method utilizing the health care network system according to another embodiment of the present invention.

The sensors 200 installed in home instruments 210, 220, 230, 240, 250, and 260 measure a user's individual biological states and transmit them to a home server 100 as shown in FIG. 6A (S110).

The home server 100 stores biological state information collected and transmitted by each biological state measuring sensor 200 individually for each user (S120). The home server 100 analyzes the user's health state from the collected biological state information and stores the result of the analysis (S130). Further, the home server 100 transmits the analyzed user's health state information together with the collected biological state information to a smart communicator 300 (S140). The user's smart communicator 300 may be located at home or outside the home, and the home server 100 utilizes a correspondingly available communication method to communicate with the smart communicator 300 according to the location of the smart communicator 300.

The smart communicator 300 displays the user's health state information received from the home server 100 on a display unit 320 and stores the information in a storage unit 390 (S150).

Additionally, the home server 100 determines whether the user has health abnormality according to the health state information analyzed in the step S130 (S160). If health abnormality is identified, the home server 100 transmits the health abnormality information to the user's smart communicator 300 (S170).

The smart communicator 300 displays and stores the health abnormality information transmitted by the home server 100 (S180). The smart communicator 300 then identifies an input of user's prescription request corresponding to the health abnormality information (S190). If an input command for a prescription request is identified, the smart communicator 300 transmits the prescription request information to the home server 100 (S210).

According to the prescription request of the smart communicator 300, the home server 100 transmits the user's health state, health abnormality, and prescription request information to a registered hospital server 500 (S220). The hospital server 500 provides a pharmaceutical prescription according to the user's health state, health abnormality, and prescription request information (S230), and transmits the corresponding prescription information to the home server 100 (S240).

The home server 100 stores the prescription information provided by the hospital server 500 (S250) and transmits the prescription information to the smart communicator 300 (S260). The smart communicator 300 displays and stores the prescription information transmitted by the home server 100 (S270).

Accordingly, reliable biological state information may be collected in real time, and a correct analysis of the health states is enabled. Further, an action corresponding to the occurrence of health abnormality may be taken promptly by measuring the user's health states without the user's consciousness in the user's daily life through transmitting such information to the user's smart communicator 300, which analyzes the user's health state if health abnormality is identified, connects to a registered hospital server 500 if there is a corresponding prescription request, and receives a pharmaceutical prescription for the user.

As apparent to those skilled in the art as explained above, the present invention may be very useful in the health care fields as identification of the user's health state in real time and early detection of health abnormality are enabled by measuring and analyzing the user's biological states with sensors in the user's daily life.

Although example embodiments of the present invention have been described in detail hereinabove, it should be understood that variations and/or modifications of the basic inventive concept herein described, which may appear to those skilled in the art, will still fall within the spirit and scope of the example embodiments of the present invention as defined in the appended claims.

What is claimed is:

1. A health care network system comprising:
a hospital server for providing a prescription based on a user's health state information, via the Internet, in response to a prescription request;
at least one biological state measuring sensor installed in at least one home instrument to detect various biological states of the user;
a home server for analyzing and transmitting the user's health state based on the detected biological states to the hospital server and receiving the prescription from the hospital server; and
a smart communicator for receiving the user's health state information and health abnormality information transmitted by the home server, transmitting the user's prescription request information to the home server in response to the prescription request, and receiving the prescription transmitted by the home server for a subsequent presentation to the user.

2. The health care network system of claim 1, wherein the home server includes:
a display unit for displaying information generated according to the operation of the home server;
a communication unit for performing a mutual communication with the at least one biological state measuring sensor, the smart communicator, and the hospital server;
a health state analyzing unit for analyzing the user's health state based on the biological states;
a control unit for receiving the prescription by connecting to the hospital server through the communication unit; and
a storage unit for storing information on the user's biological states and the prescription.

3. The health care network system of claim 2, wherein the storage unit further stores information on a pharmaceutical prescription relating to the analyzed health state.

4. The health care network system of claim 2, wherein the storage unit further stores information on a basic health care guide as a basic guide for the user's health care and conservation.

5. The health care network system of claim 4, wherein the home server further includes
a health care guide unit for comparing the result of the health state analyzed by the health state analyzing unit with basic health care information stored in the storage unit, and generating information on a health care guide for the user's health care according to the basic health care information.

6. The health care network system of claim 6, wherein the control unit transmits the health care guide information generated by the health care guide unit to the smart communicator.

7. The health care network system of claim 1, wherein the smart communicator includes:
a communication unit for performing a mutual communication with the home server in a local area and/or in a wide area;
a display unit for displaying information transmitted by the home server and information generated by the operation of the smart communicator;
a storage unit for storing the health state information and the prescription transmitted by the home server; and
a key-input unit for inputting the prescription request.

8. The health care network system of claim 7, wherein the communication unit includes at least one of the following:
an IR (infrared) communication unit for communicating at home with the home server;
a Wibro communication unit for providing a bi-directional wireless communication outside the home with the home server through a Wibro communication network;
a Zigbee module for providing a function of local area wireless communication for home automation and data network of the home server; and
a GPS (Global Positioning System) module for receiving information on longitude and latitude from satellites and identifying the user's current location.

9. The health care network system of claim 1, wherein the biological state measuring sensor includes at least one from the group consisting of:
a body weight measuring sensor, body moisture measuring sensor, body fat measuring sensor, electrocardiogram measuring sensor, blood pressure/pulse rate measuring sensor, breathing rate measuring sensor, body height measuring sensor, body temperature measuring sensor, and exercise rate measuring sensor.

10. A health care method of a health care network system utilizing a smart communicator, comprising the steps of:
measuring a user's various biological states using a plurality of biological state measuring sensors installed in home instruments;
analyzing the user's health state based on the measured biological states and identifying health abnormality;
transmitting the analyzed health state and health abnormality information to the user's smart communicator;
receiving a prescription based on the health state and health abnormality information by connecting to a registered hospital server when a request for a prescription is received from the user's smart communicator; and
transmitting the received prescription to the user's smart communicator.

11. The health care method of claim 10, further including a step of storing the analyzed health state and health abnormality information.

12. The health care method of claim 10, further including a step of storing the prescription received from the hospital server.

13. The health care method of claim 10, wherein the biological state measuring sensor includes at least one from the group consisting of: a body weight measuring sensor, body moisture measuring sensor, body fat measuring sensor, electrocardiogram measuring sensor, blood pressure/pulse rate measuring sensor, breathing rate measuring sensor, body height measuring sensor, body temperature measuring sensor, and exercise rate measuring sensor.

14. The health care method of claim 10, wherein the prescription includes a guideline for recovering from the health abnormality.

15. The health care method of claim 10, wherein the prescription includes a pharmaceutical prescription relating to the analyzed health state and health abnormality information.

16. The health care network system of claim 1, wherein the prescription includes a guideline for recovering in response to the analyzed health state.

17. The health care network system of claim 1, wherein the prescription includes a pharmaceutical prescription relating to the analyzed health state.
